(19) United States
(12) Patent Application Publication
(10) Pub. No.: US 2026/0165663 A1
(43) Pub. Date: Jun. 18, 2026

(54) MEDICAL DEVICE ASSISTANCE SUMMONS

(71) Applicant: Welch Allyn, Inc., Skaneateles Falls, NY (US)

(72) Inventors: Thomas A. Myers, Syracuse, NY (US); Rebecca Quilty-Koval, Baldwinsville, NY (US); Eric Reyer, Raleigh, NC (US); Anthony Decheek, Salt Lake City, UT (US); Patrice Etchison, Cary, NC (US); Nancy Tran, Syracuse, NY (US); Angela J. Williams, Midlothian, VA (US); Claire Ratcliffe, Coventry (GB); Hannah Drum-Paquette, Savannah, GA (US); Christian Mazzoleni, Milan (IT); Greta Magugliani, Milan (IT)

(21) Appl. No.: 19/389,470

(22) Filed: Nov. 14, 2025

Related U.S. Application Data
(60) Provisional application No. 63/720,260, filed on Nov. 14, 2024.

(51) Int. Cl. A61B 5/00

(52) U.S. Cl. CPC A61B 5/746; A61B 5/0022; A61B 5/7275; A61B 5/7282; A61B 5/7435

(57) ABSTRACT

A medical device includes one or more modules configurable to connect to one or more sensors for capturing one or more physiological variable measurements. The medical device includes a display screen for displaying the physiological variable measurements. The medical device triggers an alarm based on the physiological variable measurements captured by the one or more sensors. The medical device displays a first option on the display screen to suppress the alarm and a second option on the display screen to submit a request for assistance.

### CROSS-REFERENCE TO RELATED APPLICATION(S)

This application claims the benefit of U.S. Provisional Application No. 63/720,260, filed Nov. 14, 2024, the disclosure of which is hereby incorporated by reference in its entirety.

### BACKGROUND

Alarms triggered by medical devices are often silenced or dismissed by healthcare professionals to temporarily disable flashing lights and/or mute sounds that are emitted by the medical devices. While alarms are essential for alerting the healthcare professionals to adverse health conditions and safety issues, excessive or non-urgent alarms can lead to alarm fatigue, which can cause the healthcare professionals to become desensitized to the alarms over time such that they may not respond promptly to critical alarms. Alarm silencing is often employed to reduce noise and other conditions that can cause alarm fatigue within a clinical setting.

Further, healthcare professionals who respond to alarms triggered on medical devices will often need assistance such as help with one or more tasks or additional medical equipment to address the conditions that are causing the alarms to trigger. Such assistance is often requested by a device separate from the medical device triggering the alarm. This can make it challenging for a healthcare professional to both silence the alarm on the medical device and request the assistance to address the condition triggering the alarm on the medical device.

### SUMMARY

In general terms, the present disclosure relates to a medical device. In one possible configuration, the medical device provides both a first option to suppress an alarm triggered on the medical device and a second option to submit a request for assistance. Various aspects are described in this disclosure, which include, but are not limited to, the following aspects.

One aspect relates to a medical device, comprising: one or more modules configurable to connect to one or more sensors for capturing one or more physiological variable measurements; a display screen for displaying the physiological variable measurements; at least one processing device; and at least one memory device storing software instructions that, when executed by the at least one processing device, cause the at least one processing device to: trigger an alarm based on the physiological variable measurements captured by the one or more sensors; and display a first option on the display screen to suppress the alarm and a second option on the display screen to submit a request for assistance.

Another aspect relates to a method of monitoring a patient, the method comprising: receiving measurements of a physiological variable; triggering an alarm based on the measurements of the physiological variable; and displaying a first option on a display screen to suppress the alarm and a second option on the display screen to submit a request for assistance.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 is a front isometric view of a medical device that can be used to monitor one or more physiological variables of a patient.

FIG. 2 is a rear isometric view of the medical device of FIG. 1.

FIG. 3 is a front view of the medical device of FIG. 1.

FIG. 4 is a bottom view of the medical device of FIG. 1.

FIG. 5 schematically illustrates an example of the medical device of FIG. 1.

FIG. 6 schematically illustrates an example of a method of monitoring a patient that can be performed by the medical device of FIG. 1.

FIG. 7 illustrates an example of a graphical user interface displayed on a display screen of the medical device of FIG. 1 in accordance with the method of FIG. 6.

FIG. 8 illustrates an example of a graphical user interface displayed on the display screen of the medical device of FIG. 1, the graphical user interface including an assistance shortcut.

FIG. 9 illustrates an example of a graphical user interface displayed on the display screen of the medical device of FIG. 1, the graphical user interface including assistance options displayed in response to a selection of the assistance shortcut in the graphical user interface of FIG. 8.

FIG. 10 illustrates an example of a workflow that includes first and second graphical user interfaces displayed on the display screen of the medical device of FIG. 1, the first graphical user interface displays an alarm message while in an unattended mode, and the second graphical user interface displays the alarm message while in an attended mode.

### DETAILED DESCRIPTION

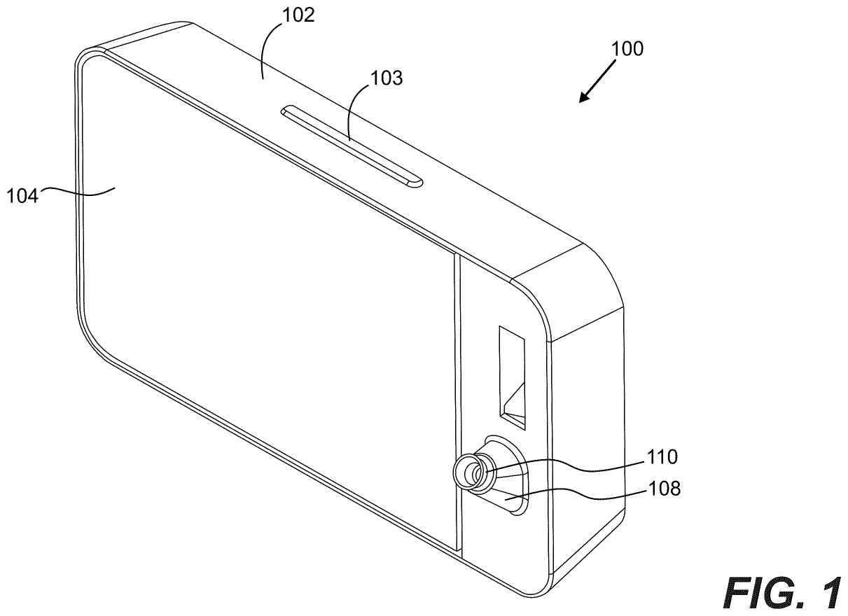

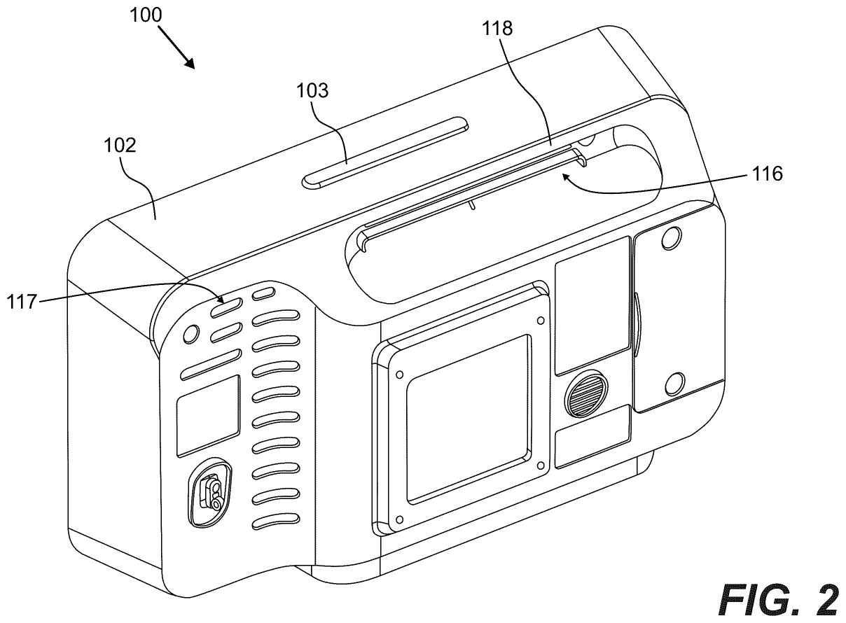

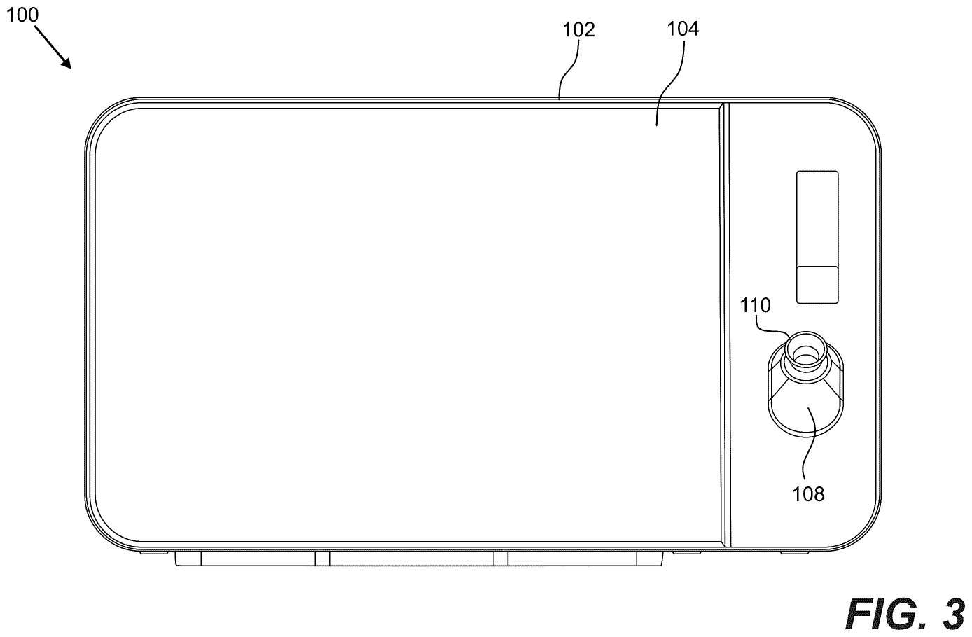

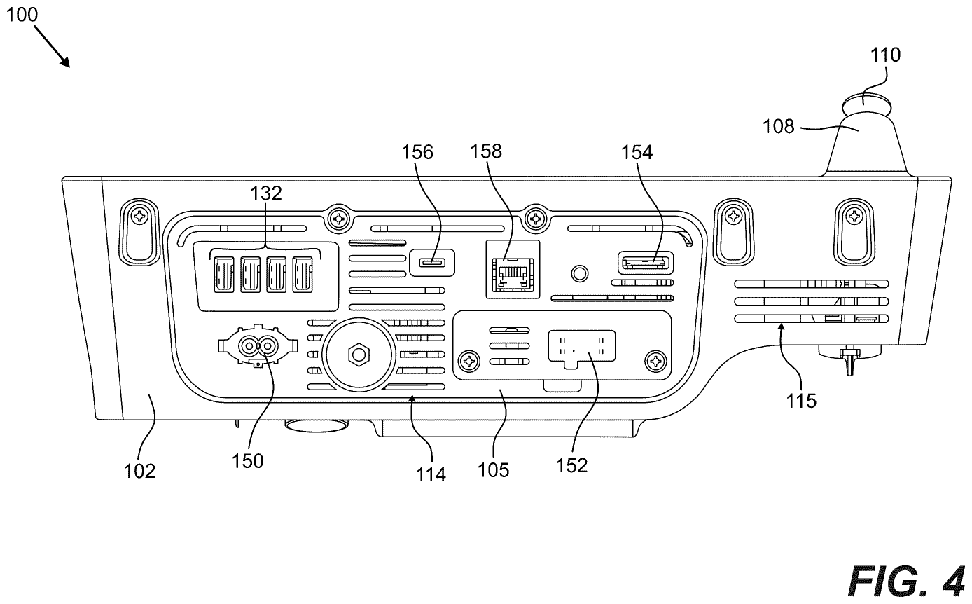

FIG. 1 is a front isometric view of a medical device 100 that can be used to monitor one or more physiological variables of a patient. FIG. 2 is a rear isometric view of the medical device 100. FIG. 3 is a front view of the medical device 100. FIG. 4 is a bottom view of the medical device 100. As shown in FIGS. 1-4, the medical device 100 includes a housing 102, and a display screen 104 mounted on a front portion of the housing 102. The display screen 104 is a touch sensitive touchscreen that receives inputs from a user of the medical device 100 such as a caregiver, a physician, a clinician, a nurse, or other trained healthcare professional.

The medical device 100 is designed for use in a clinical setting. For example, the medical device 100 can be used to continuously monitor one or more physiological variables of a patient admitted to a medical facility such as a hospital, medical clinic, and the like. Additionally, the medical device 100 can operate as a spot monitor for episodic monitoring of one or more physiological variables of a plurality of patients in the medical facility.

The medical device 100 includes one or more modules configurable to connect to one or more sensors that can be used to measure the one or more physiological variables including, without limitation, blood pressure, blood oxygen saturation (SpO2), pulse rate, body temperature, and respiration rate. The medical device 100 can include a thermometer module 108 to measure body temperature, a module configured to connect to a non-invasive blood pressure cuff to measure systolic and diastolic blood pressure, a module configured to connect to a pulse oximeter to measure blood oxygen saturation and pulse rate, and can include additional types of modules and sensors for capturing additional types of physiological variable measurements.

The sensors can include cables that terminate into connectors that plug into various ports of the modules included on the housing 102 of the medical device 100 for transmission of data to the medical device 100. As shown in FIG. 4, the housing 102 includes a first port 150 for connecting the non-invasive blood pressure cuff and a second port 152 for connecting the pulse oximeter. Optionally, the medical device 100 can include a third port 154 for connecting the medical device 100 to a printer. Also, the medical device 100 can include one or more universal serial bus (USB) ports 132, a USB-C port 156, and a registered jack (RJ) interface 158. In some examples, at least some of the sensors wirelessly transmit data to the medical device 100 via a wireless connection established through Wi-Fi, Bluetooth, or another wireless protocol.

In the example shown in FIGS. 1-4, the thermometer module 108 is integrated with the medical device 100. The thermometer module 108 includes a port 110 for housing a handheld probe that can be orally inserted into the mouth of a patient to take a temperature reading. Alternatively, the housing 102 of the medical device 100 can be configured to house a handheld probe that can be inserted into an ear of the patient to take a temperature reading.

The medical device 100 receives the data from the sensors for processing. For example, the medical device 100 can generate numerical values and/or waveforms for the one or more physiological variables based on the data received from the sensors. The medical device 100 can display the numerical values and/or waveforms of the one or more physiological variables on the display screen 104. Also, the medical device 100 can generate alarms based on the data received from the sensors when an alarm condition is detected for a patient who is being monitored by the medical device 100.

The alarms generated by the medical device 100 can include a visual alarm displayed on the display screen 104 and an audible alarm emitted by a speaker of the medical device 100. An additional visual alarm can be displayed on an illumination unit 103, which is positioned on the top portion of the housing 102 of the medical device 100. The illumination unit 103 allows for distant viewing of critical alarms. The critical alarms generated on the illumination unit 103 can be viewed across a 360 degree field of view around the medical device 100. In some examples, both a visual alarm is displayed on the display screen 104 and a further visual alarm is generated on the illumination unit 103 to indicate a critical status of a detected alarm condition.

The illumination unit 103 can include one or more light-emitting diodes (LEDs) that emit light that is visible through a translucent cover. As an illustrative example, the illumination unit 103 can emit light having a color (e.g., yellow or red) based on a severity of the alarm. Additionally, or alternatively, the illumination unit 103 can emit light flashes based on a predetermined pattern associated with the severity of the alarm (e.g., an increased blinking rate to indicate severe conditions and a decreased blinking rate to indicate less severe conditions).

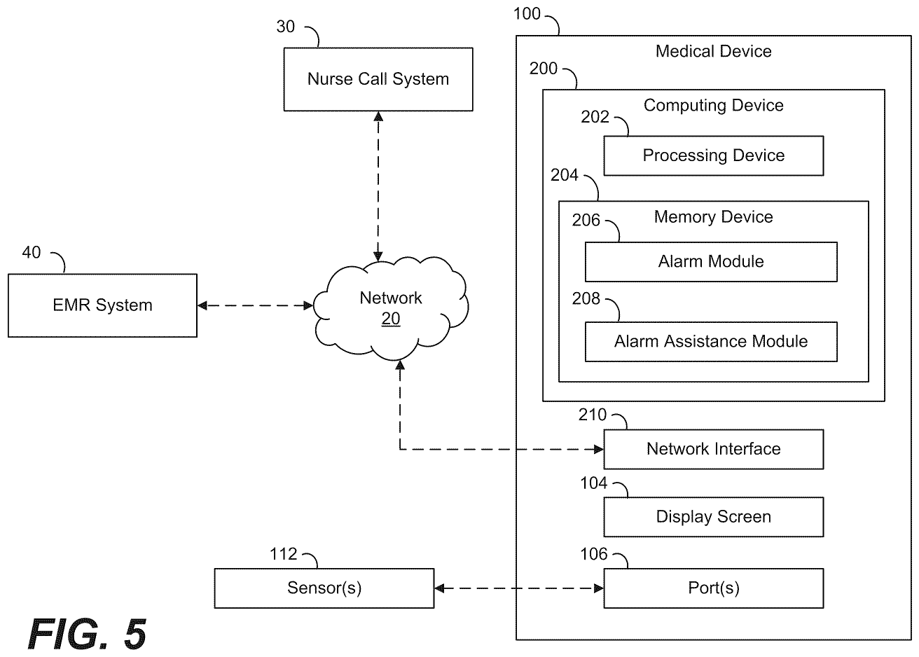

FIG. 5 schematically illustrates an example of the medical device 100. As shown in FIG. 5, the medical device 100 includes a computing device 200 having at least one processing device 202 and at least one memory device 204 that stores software instructions that, when executed by the at least one processing device 202, cause the at least one processing device 202 to perform the various aspects, functions, and operations described herein.

The at least one processing device 202 is an example of a processing unit such as a central processing unit (CPU). The at least one processing device 202 can include one or more CPUs. In some examples, the at least one processing device 202 includes one or more digital signal processors, field-programmable gate arrays, and/or other types of electronic circuits.

The at least one memory device 204 is an example of a computer-readable data storage device that operates to store data and instructions for execution by the at least one processing device 202. The at least one memory device 204 includes computer-readable media, which includes any media that can be accessed by the at least one processing device 202. The computer-readable media can include computer-readable storage media and computer-readable communication media. The computer-readable storage media includes volatile and nonvolatile, removable and non-removable media implemented in any device that can store information such as computer-readable instructions, data structures, program modules, or other data. The computer-readable storage media can include random access memory, read only memory, electrically erasable programmable read only memory, flash memory, and other memory technology, including any medium that can be used to store information that can be accessed by the at least one processing device 202. The computer-readable storage media is non-transitory.

The computer-readable communication media embodies computer-readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. The computer-readable communication media can include wired media such as a wired network or direct-wired connection, and wireless media such as acoustic, radio frequency, infrared, and other wireless media. Combinations of any of the above are within the scope of computer-readable media.

As shown in FIG. 5, the at least one memory device 204 stores an alarm module 206 which can include one or more algorithms for determining whether to trigger an alarm based on the physiological variable measurements captured by one or more sensors 112 that can plug into one or more ports 106 of the medical device 100 (e.g., the first port 150, the second port 152, etc.), and/or that can wirelessly transmit data to the medical device 100, and/or that can be integrated with the medical device 100. In accordance with the examples described above, the one or more sensors 112 can include a thermometer probe to measure body temperature, a non-invasive blood pressure (NIBP) cuff to measure systolic and diastolic blood pressure, a pulse oximeter to measure blood oxygen saturation and pulse rate, and additional types of sensors for capturing additional types of physiological variable measurements.

The at least one memory device 204 further stores an alarm assistance module 208 which can display one or more options on the display screen 104 of the medical device 100 to request assistance to ameliorate a condition causing the alarm module 206 to trigger the alarm.

The medical device 100 includes a network interface 210 that facilitates connection to the network 20 that can include any type of wired or wireless connections or any combinations thereof. The network interface 210 can include wired interfaces and/or wireless interfaces. For example, the network interface 210 can be used to wirelessly connect the medical device 100 to the network 20 such as through Wi-Fi and the like. Alternatively, or additionally, the network interface 210 can be used to connect the medical device 100 to the network 20 using wired connections such as through Ethernet or Universal Serial Bus (USB) cables.

As shown in FIG. 5, the medical device 100 communicates with a nurse call system 30 via the network 20. Selections of the one or more options displayed on the display screen 104 of the medical device 100 by the alarm assistance module 208 can be sent to the nurse call system 30 via the network 20 to route requests for assistance to fix and/or ameliorate a condition causing the alarm module 206 to trigger an alarm on the medical device 100.

The medical device 100 also communicates with an electronic medical record (EMR) system 40 via the network 20. The physiological measurements captured by the one or more sensors 112 are sent to the EMR system 40 via the network 20 for storage in an electronic medical record (EMR) (alternatively termed electronic health record (EHR)) of a patient. The EMR operates to manage the patient's medical history and information. The EMR system 40 can be operated by a healthcare service provider such as a hospital or medical clinic to maintain a plurality of EMRS for a plurality of patients of the healthcare service provider.

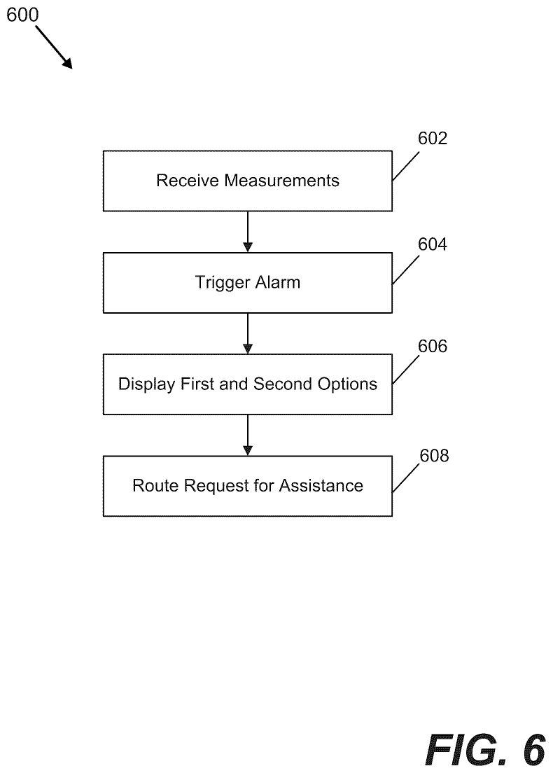

FIG. 6 schematically illustrates an example of a method 600 of monitoring a patient that can be performed by the medical device 100. The method 600 can improve the ability to respond to a medical emergency that is causing the alarm module 206 to trigger an alarm on the medical device 100. As shown in FIG. 6, the method 600 includes an operation 602 of receiving measurements of one or more physiological variables from the one or more sensors 112 that connect with or otherwise integrate with the medical device 100. As an illustrative example, operation 602 can include receiving measurements of non-invasive blood pressure (systolic and diastolic) from a blood pressure sensor, blood oxygen saturation (SpO2) and pulse rate from a pulse oximeter, body temperature from a thermometer, heart rate and/or respiration rate from sensors embedded in a patient support apparatus such as a hospital bed.

In some examples, operation 602 can include calculating one or more composite scores using the measurements of the one or more physiological variables. Illustrative examples of the one or more composite scores can include an early warning score (EWR), modified early warning score (MEWS), quick sequential organ failure assessment score (qSOFA score), systemic inflammatory response syndrome (SIRS score), and other composite scores.

The method 600 includes an operation 604 of triggering an alarm based on the measurements of the one or more physiological variables received in operation 602. Operation 604 can be performed by the alarm module 206. In operation 604, the alarm can be triggered based on the measurements of the one or more physiological variables being above an upper threshold or below a lower threshold. Alternatively, or additionally, operation 604 can include triggering the alarm when a composite score exceeds an upper threshold such as when an early warning score (EWR) exceeds an upper threshold such that the patient is at risk of deterioration.

The method 600 includes an operation 606 of displaying a first option on the display screen 104 to suppress the alarm triggered in operation 604, and also displaying one or more second options on the display screen 104 to submit a request for assistance such as to fix and/or ameliorate a condition that is causing the alarm to be triggered in operation 604. Operation 606 can be performed by the alarm assistance module 208. During operation 606, the first and second options can be displayed simultaneously on the display screen 104 of the medical device 100.

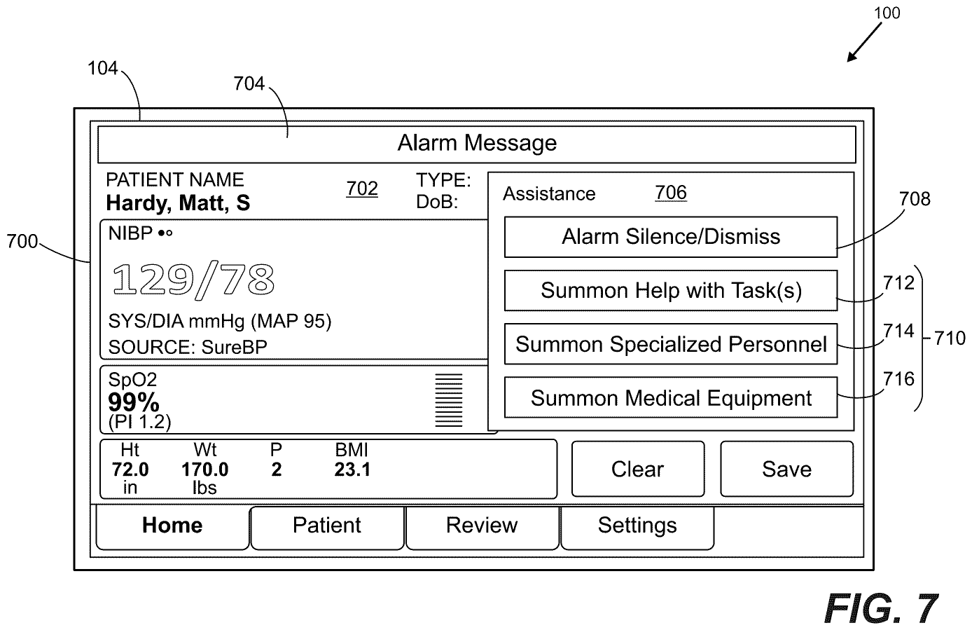

FIG. 7 illustrates an example of a graphical user interface 700 displayed on the display screen 104 of the medical device 100 in accordance with the operations of the method 600 of FIG. 6. The graphical user interface 700 includes a monitoring area 702 that displays measurements of one or more physiological variables such as non-invasive blood pressure (systolic and diastolic), blood oxygen saturation (SpO2), pulse rate, respiration rate, body temperature, and other data such as patient demographic data (e.g., patient name, date of birth, patient ID number), and other measurements such as the height, weight, and body mass index (BMI) of a patient who is being monitored by the medical device 100.

The graphical user interface 700 further includes an alarm message 704 that is generated in response to an alarm being triggered by the measurements of the one or more physiological variables displayed in the monitoring area 702. The alarm message 704 can include a short message explaining why the alarm is triggered (e.g., patient is at risk for septic shock because blood pressure is below lower limit). The alarm message 704 can be generated by the alarm module 206. The alarm message 704 can be generated in operation 604 of the method 600. In the example illustrated in FIG. 7, the alarm message 704 is displayed across a top of the graphical user interface 700 such that it can be easily viewed.

The graphical user interface 700 further includes an assistance request area 706 that is generated by the alarm assistance module 208. The assistance request area 706 can be generated in operation 606 of the method 600. In this illustrative example, the assistance request area 706 includes a first option 708 to silence or dismiss the alarm. The first option 708 when selected disables flashing lights (e.g., on the illumination unit 103 and the display screen 104) and mutes sounds that are emitted by the medical device 100. In some examples, selection of the first option 708 causes the alarm message 704 to disappear from the graphical user interface 700.

The assistance request area 706 includes a second option 710 that includes one or more assistance options 712-716 to request assistance such as to help fix or ameliorate a condition causing the alarm to be triggered on the medical device 100. The one or more assistance options 712-716 are customizable such as based on the medical facility where the medical device 100 is deployed.

The graphical user interface 700 allows selection of the first option 708, or selection of the second option 710, or selection of both the first option 708 and the second option 710. In some examples, selection of the second option 710 automatically causes the alarm to be silenced or dismissed such that it is not necessary to select the first option 708. In further examples, the graphical user interface 700 allows selection of more than one of the assistance options 712-716.

In the example illustrated in FIG. 7, the second option 710 includes a first assistance option 712 to request help with one or more types of tasks. For example, upon selection of the first assistance option 712, the graphical user interface 700 displays a drop down menu to show a list of tasks such as to help ambulate the patient (e.g., help patient ambulate to bathroom), help turn the patient on a patient support device such as a hospital bed, and help with other tasks.

The second option 710 can also include a second assistance option 714 to request one or more types of specialized personnel. Upon selection of the second assistance option 714, the graphical user interface 700 displays a drop down menu to show a list of specialized personnel such as particular types of physicians, medical professionals, or groups of medical professionals such as a rapid response team who can be summoned to immediately assess and treat the patient when the patient demonstrates signs of imminent clinical deterioration.

The second option 710 can also include a third assistance option 716 to request one or more types of medical equipment. Upon selection of the third assistance option 716, the graphical user interface 700 displays a drop down menu of a list of medical equipment. In some instances, more than one piece of medical equipment can be selected from the drop down menu.

In some examples, the one or more assistance options 712-716 are customized based on the alarm triggered on the medical device 100. For example, the second option 710 can include one or more additional types of assistance options for display on the graphical user interface 700 based on the type of alarm triggered on the medical device 100. In further examples, the second option 710 includes a fewer number of assistance options for display on the graphical user interface 700 based on the type of alarm triggered on the medical device 100. For example, when an alarm is triggered because the patient is detected as attempting to leave their bed when they are not authorized to do so without assistance, the graphical user interface 700 displays the first assistance option 712 to request help with ambulating the patient out of their bed, but does not display the second assistance option 714 or the third assistance option 716.

In further examples, the drop down menus that are displayed upon selection of the first assistance option 712, or the second assistance option 714, or the third assistance option 716 are customized based on the alarm triggered on the medical device 100. For example, the tasks displayed for selection under the first assistance option 712 are customized based on the alarm triggered on the medical device 100. Similarly, the specialized personnel displayed for selection under the second assistance option 714 are customized based on the alarm triggered on the medical device 100. Similarly, the medical equipment displayed for selection under the third assistance option 716 are customized based on the alarm triggered on the medical device 100.

In some examples, the one or more assistance options 712-716 are customized based on the location of the medical device 100. For example, the second option 710 can include one or more additional types of assistance options for display on the graphical user interface 700 based on the location of the medical device 100. In further examples, the second option 710 includes a fewer number of assistance options for display on the graphical user interface 700 based on the location of the medical device 100. For example, when the medical device 100 is located inside an emergency room department (EMR), the graphical user interface 700 displays the second assistance option 714 and the third assistance option 716, but not the first assistance option 712.

In further examples, the drop down menus that are displayed upon selection of the first assistance option 712, or the second assistance option 714, or the third assistance option 716 are customized based on the location of the medical device 100. For example, the tasks displayed for selection under the first assistance option 712 are customized based on the location of the medical device 100. Similarly, the specialized personnel displayed for selection under the second assistance option 714 are customized based on location of the medical device 100. Similarly, the pieces of medical equipment displayed for selection under the third assistance option 716 are customized based on the location of the medical device 100.

Referring back to FIG. 6, the method 600 can further include an operation 608 of routing the request for assistance to one or more caregivers. In some examples, operation 608 includes using the nurse call system 30 to route one or more requests for assistance to the one or more caregivers. For example, the one or more requests for assistance can be communicated from the medical device 100 to the nurse call system 30 via the network 20 (see FIG. 5). Once the nurse call system 30 receives the one or more requests, the nurse call system 30 routes the one or more requests for assistance to mobile devices carried by the caregivers (e.g., smartphones, tablet computers, and the like) and/or routes the one or more requests for assistance to one or more workstations such as desktop personal computers and/or wall mounted display screens in an area within the medical facility such as a nurses'station within a hospital.

In some examples, the request for assistance includes a location of the medical device 100. Including the location of the medical device 100 along with the request for assistance allows the caregivers to know where to direct the requested tasks, personnel, or equipment.

The location of the medical device 100 can be based on an association of the medical device 100 with a patient. For example, when the patient is admitted to the medical facility, the patient is assigned to a room or other location within the medical facility. This information can be maintained by an admission, discharge, and transfer (ADT) system that tracks the patient from the time they arrive until they leave. Further, the medical device 100 is assigned to the patient or can otherwise be designated as monitoring the patient such that the location of the medical device 100 is determined to be the same as that of the patient.

Alternatively, the location of the medical device 100 can based on a location identification device attached to the medical device 100. For example, the medical device 100 can be equipped with a global positioning system (GPS) tracking device. Alternatively, the medical device 100 can emit a Bluetooth signal that privately and securely connects to any device in the network 20, and then the location of the medical device 100 is triangulated by the connections to the other devices. In further examples, the location of the medical device 100 can be determined by a real-time locating system (RTLS) that utilizes readers dispersed throughout the medical facility to read a tag attached to the medical device 100 to determine the location of the medical device 100. Illustrative examples of the types of tags that can be attached to the medical device 100 can include radio-frequency identification (RFID) tags, near-field communication (NFC) tags, ultra-wideband (UWB) tags, and other types of tags that can emit wireless signals that can be detected by readers dispersed within the medical facility.

Alternatively, the location of the medical device 100 can based on an input received on the display screen 104 of the medical device 100. For example, the graphical user interface 700 can display an input area that allows a user of the medical device 100 to manually enter the location of the medical device 100 or another location where to direct the request for assistance.

In further examples, the graphical user interface 700 can display the location of the medical device 100 determined from any of the abovementioned options, and request the user of the medical device 100 to confirm the location. If the user declines to confirm the location, the user can manually enter a new location for where to direct the request for assistance.

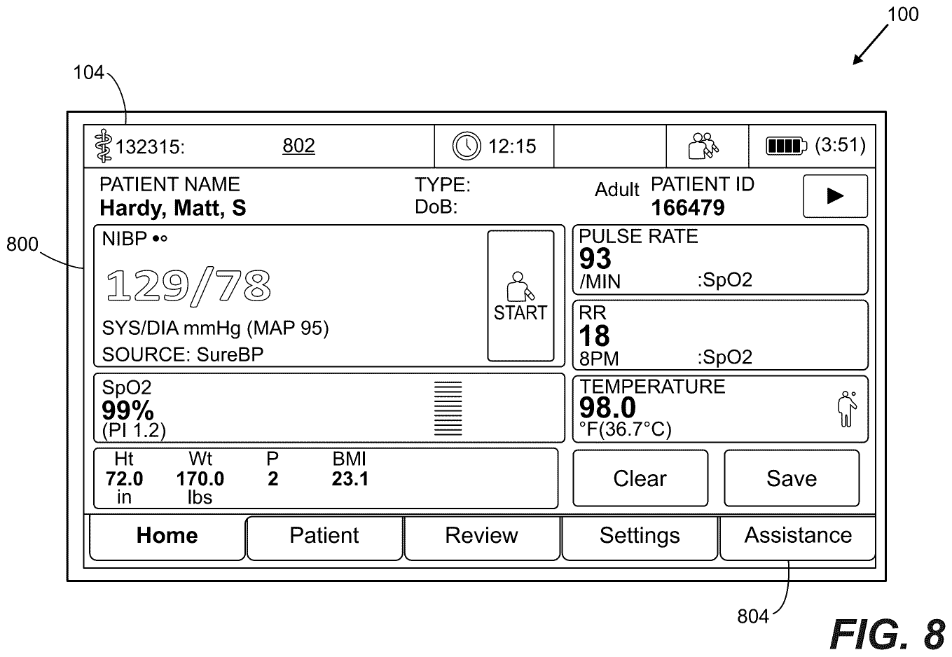

FIG. 8 illustrates another example of a graphical user interface 800 displayed on the display screen 104 of the medical device 100. In this example, the graphical user interface 800 includes a monitoring area 802 that is displayed when an alarm is not triggered on the medical device 100. The graphical user interface 800 further includes an assistance shortcut 804 which when selected causes the display screen 104 to display the assistance request area when an alarm is not triggered on the medical device 100. The one or more assistance options displayed in the assistance request area are customizable such as based on the medical facility where the medical device 100 is deployed.

In the illustrative example shown in FIG. 8, the monitoring area 802 displays measurements of one or more physiological variables of a patient who is being monitored by the medical device 100 such as non-invasive blood pressure (systolic and diastolic), blood oxygen saturation (SpO2), pulse rate, respiration rate, body temperature, and other data such as patient demographic data (e.g., patient name, date of birth, patient ID number), and other measurements such as the height, weight, and body mass index (BMI).

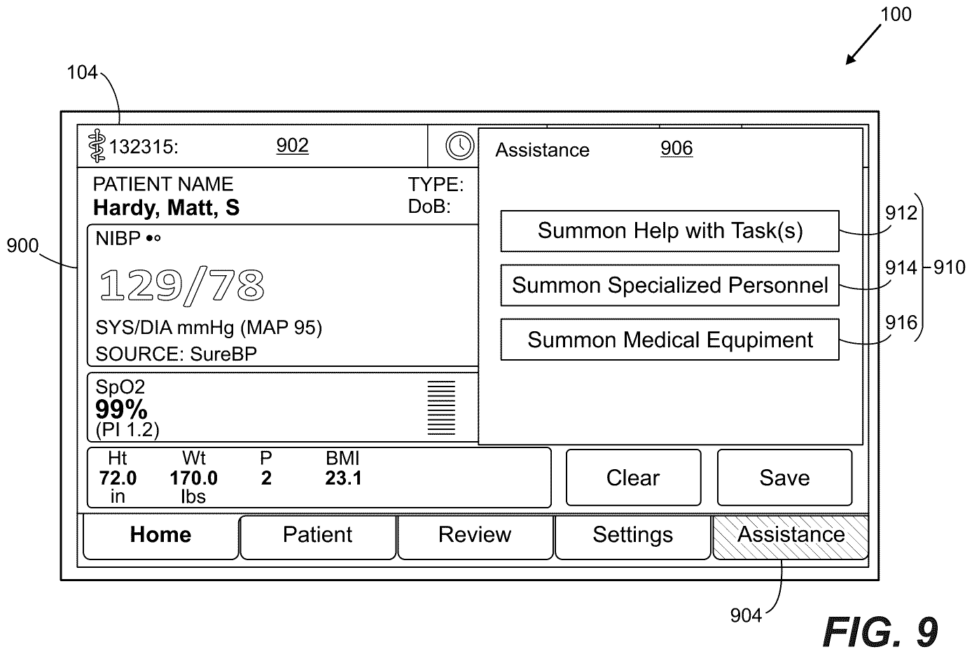

FIG. 9 illustrates an example of a graphical user interface 900 displayed on the display screen 104 of the medical device 100, the graphical user interface 900 including an assistance request area 906 displayed in response to a selection of the assistance shortcut 804 in the graphical user interface 800 of FIG. 8. The assistance request area 906 is similar to the assistance request area 706 of FIG. 7 except the assistance request area 906 does not include a first option to silence or dismiss an alarm since one is not being triggered on the medical device 100. Instead, the assistance request area 906 only includes the second option 910 that can include one or more assistance options such as a first assistance option 912 to request help with one or more types of tasks, a second assistance option 914 to request one or more types of specialized personnel, and/or a third assistance option 916 to request one or more types of medical equipment. In this manner, the user of the medical device 100 can send a request for assistance to one or more caregivers when an alarm is not being triggered on the medical device 100.

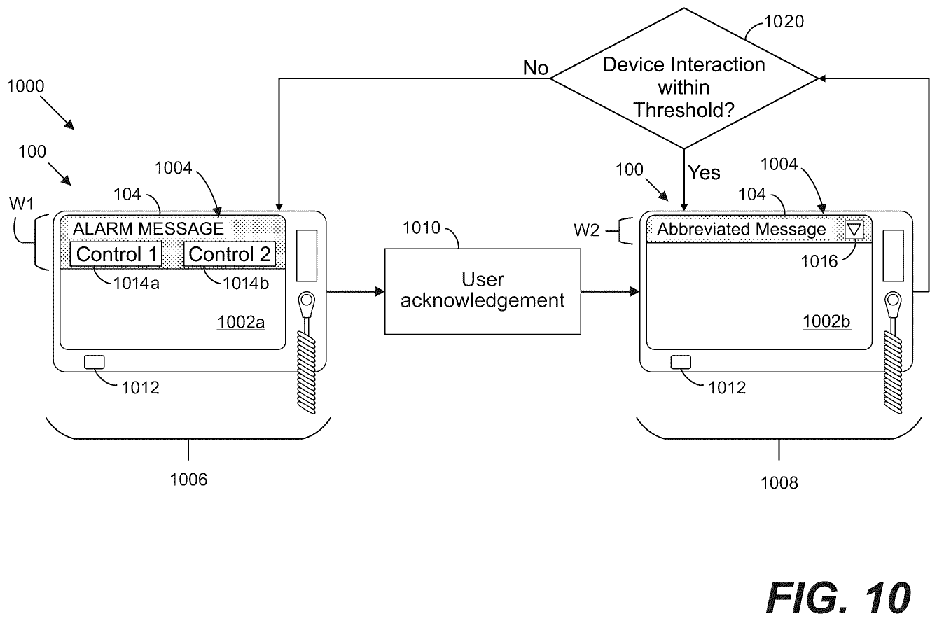

FIG. 10 illustrates an example of a workflow 1000 that includes first and second graphical user interfaces 1002a, 1002b displayed on the display screen 104 of the medical device 100. The first graphical user interface 1002a displays an alarm message 1004 while in an unattended mode 1006. The second graphical user interface 1002b displays the alarm message 1004 while in an attended mode 1008. In the unattended mode 1006, the medical device 100 displays the alarm message 1004 in response to an alarm triggered by the physiological variable measurements captured by the one or more sensors 112 communicatively coupled to the medical device 100. In accordance with the examples described above, the one or more sensors 112 can include a thermometer probe to measure body temperature, a non-invasive blood pressure (NIBP) cuff to measure systolic and diastolic blood pressure, a pulse oximeter to measure blood oxygen saturation and pulse rate, and additional types of sensors for capturing additional types of physiological variable measurements.

Upon receipt of an acknowledgement 1010 of the alarm message 1004, the medical device 100 switches from the unattended mode 1006 to the attended mode 1008. The acknowledgement 1010 can include selection of a control or input included within the alarm message 1004 displayed on the display screen 104 such as to suppress, silence, or pause the alarm that triggered the display of the alarm message 1004 on the medical device 100. In some examples, the acknowledgement 1010 includes a selection of the alarm message 1004 itself.

In alternative examples, the acknowledgement 1010 includes detection of a presence of a user such as a caregiver. The presence of the user can be detected by a sensor 1012 on the medical device 100 that detects a signal from a device worn by the user. The sensor 1012 can include a transceiver such as a radio frequency identification (RFID) reader that emits radio waves and receives a signal from an RFID tag worn by the user. The RFID tag can include a passive antenna, a semi-passive antenna, or an active antenna.

In yet further examples, the sensor 1012 can include a presence detection sensor that detects whether a person such as a user is within a certain distance of the medical device 100. The presence detection sensor detects changes in an environment in front of the medical device 100 that indicates when someone enters or occupies a monitored space by sensing motion, heat, distance, or field disturbance. For example, the presence detection sensor can include a radar sensor that sends out radio waves and detects reflections from moving or stationary objects; a capacitive sensor or an inductive sensor that detects changes in electrical or magnetic fields caused by nearby conductive or dielectric objects; an infrared (PIR) sensors that detects changes in infrared radiation (i.e., due to body heat); or an ultrasonic sensor that emits sound waves and measure how long it takes for the echo to return and when the echo pattern changes.

When in the unattended mode 1006, the alarm message 1004 has a first size on the first graphical user interface 1002a. When in the attended mode 1008, the alarm message 1004 has a second size on the second graphical user interface 1002b. As shown in FIG. 10, the first size of the second size of the alarm message 1004 in the unattended mode 1006 is larger than the second size of the alarm message 1004 in the unattended mode 1006. For example, the alarm message 1004 is displayed across a top of the first and second graphical user interfaces 1002a, 1002b. When in the unattended mode 1006, the alarm message 1004 has a first width W1. When in the attended mode 1008, the alarm message 1004 has a second width W2. As shown in FIG. 10, the first width W1 is larger than the second width W2 such that it can be easily viewed.

The two stage alarm messaging shown in the workflow 1000 provides a technical advancement where the alarm message 1004 in the unattended mode 1006 is presented in an enlarged format to enhance readability and identification of the alarm message 1004 such as when a caregiver walks by the medical device 100 and glances at the display screen 104. The larger size of the alarm message 1004 when in the unattended mode 1006 improves recognition or identification of the alarm message 1004. For example, the larger size of the alarm message 1004 when in the unattended mode 1006 enables the alarm message 1004 to have a larger font size. Also, the alarm message 1004 in the unattended mode 1006 can have an extended length and include additional information and more details than the alarm message 1004 in the attended mode 1008. For example, the alarm message 1004 in the unattended mode 1006 can include dynamic links within the displayed information to access additional information, which can include information stored locally on the medical device 100, or stored elsewhere such as on external servers. Accordingly, the alarm message 1004 in the attended mode 1008 can be an abbreviated version of the alarm message 1004 in the unattended mode 1006.

Additionally, the larger size of the alarm message 1004 when in the unattended mode 1006 enables the medical device 100 to present additional controls for selection by a user where such additional controls are not displayed on the alarm message 1004 when in the attended mode 1008 due to its smaller size. In the illustrative example of FIG. 10, the alarm message 1004 includes a first control 1014a and a second control 1014b that are each selectable on the display screen 104 to control one or more operations, functions, and/or features of the medical device 100. In some examples, the extended information and controls that are included in the alarm message 1004 in the unattended mode 1006 are preselected based on the condition that triggered the alarm such that the extended information and controls are relevant for immediate review and/or selection by a user at the moment the alarm is triggered on the medical device 100.

As shown in FIG. 10, the alarm message 1004 in the attended mode 1008 does not include the first control 1014a and a second control 1014b. Instead, the alarm message 1004 in the attended mode 1008 includes a control 1016 that when selected expands the size of the alarm message 1004 to have the same size or a similar size as when in the unattended mode 1006. Accordingly, selection of the control 1016 expands the size of the alarm message to increase the font size of the alarm message, to have the extended length that includes the additional information and details, and/or to include the first and second controls 1014a, 1014b. The ability to enlarge or reduce the size of the alarm message 1004 provides flexibility to review the extended alarms data and selectable controls after initial acknowledgement.

Additionally, the smaller size of the alarm message 1004 when in the attended mode 1008 means that it occupies less space on the second graphical user interface 1002b. This prevents information such as the physiological variable measurements captured by the one or more sensors 112 from being obscured or hidden as a result of the display of the alarm message 1004. Thus, a further technical advantage includes displaying additional information on the second graphical user interface 1002b that is not available for viewing when in the unattended mode 1006 due to the smaller size of the alarm message 1004 when in the attended mode 1008. Another technical advantage includes maintaining the confidentiality of protected health information (PHI) by obscuring or hiding the information when in the unattended mode 1006 due to the larger size of the alarm message 1004 when in the unattended mode 1006.

As further shown in FIG. 10, the workflow 1000 can further include an operation 1020 of determining whether an interaction with the medical device 100 is detected within a predetermined period of time. Examples of interactions can include touching the display screen 104 such as to select a control and/or to navigate through one or more graphical user interfaces displayed on the display screen 104. In some examples, an interaction is the presence of the user in proximity to the medical device 100 such as based on the signals captured by the sensor 1012. As an illustrative example, the predetermined period of time can include 30 seconds, 60 seconds, or other period of time. The predetermined period of time can be adjustable.

When an interaction is detected within the predetermined period of time (i.e., “Yes” in operation 1020), the workflow 1000 continues to display the alarm message 1004 in the attended mode 1008. When an interaction is not detected within the predetermined period of time (i.e., “No” in operation 1020), the workflow 1000 causes the medical device 100 to revert to displaying the alarm message 1004 in the unattended mode 1006.

This technical disclosure addresses a technical problem in medical monitoring systems where existing spot monitors lack integrated assistance request functionality. Conventional spot monitors are limited to basic alarm generation and suppression capabilities, without any technical means to communicate contextual alarm information or assistance requests to external clinical systems. This technical limitation requires separate communication infrastructure to handle assistance requests, creating a fragmented system architecture where alarm data and assistance coordination operate through disconnected technical pathways.

This technical disclosure provides a technical solution to the aforementioned technical problem by integrating the alarm assistance module 208 directly within the computing architecture of the medical device 100 alongside the alarm module 206. This represents a technical advancement where the medical device 100 itself gains the technical capability to process, format, and transmit assistance requests rather than relying on external systems.

Additionally, the technical solution provided by the medical device 100 is enabled by the network interface 210 which provides direct communication functionality with the nurse call system 30. Further, the medical device 100 includes location data in assistance requests through integrated location determination capabilities (GPS, Bluetooth triangulation, RTLS integration, or ADT system connectivity), which further enhances the technical solution. The technical capabilities of the medical device 100 are expanded through the alarm assistance module 208 that automatically processes alarm trigger data to generate contextually-appropriate assistance options enabling the device itself to technically correlate alarm conditions with relevant assistance categories (tasks, personnel, equipment) without requiring external processing.

The technical solution provided by the medical device 100 additionally implements simultaneous display capabilities on the display screen 104 where both alarm suppression and assistance request interfaces operate through the same display. This represents a technical improvement where display processing handles multiple concurrent interface functions.

Additionally, the medical device 100 gains technical capabilities from composite score calculation (e.g., EWR, MEWS, qSOFA) that directly integrate with the alarm assistance module 208, enabling more sophisticated technical alarm conditions that automatically trigger the integrated assistance request capabilities provided by the alarm assistance module 208.

In view of the foregoing, the medical device 100 provides measurable technical improvements to medical device functionality by: reducing the number of device interfaces healthcare professionals must navigate during emergencies; providing automated, context-sensitive assistance options that improve response accuracy; integrating location services to enable precise routing of assistance requests; and expanding the functional capabilities of spot monitors beyond basic monitoring to include clinical workflow integration.

The various embodiments described above are provided by way of illustration only and should not be construed to be limiting in any way. Various modifications can be made to the embodiments described above without departing from the true spirit and scope of the disclosure.

What is claimed is:

1. A medical device, comprising:
one or more modules configurable to connect to one or more sensors for capturing one or more physiological variable measurements;
a display screen for displaying the physiological variable measurements;
at least one processing device; and
at least one memory device storing software instructions that, when executed by the at least one processing device, cause the at least one processing device to:
trigger an alarm based on the physiological variable measurements captured by the one or more sensors; and
display a first option on the display screen to suppress the alarm and a second option on the display screen to submit a request for assistance.

2. The medical device of claim 1, wherein the display screen is a touchscreen that allows selection of the first option, or the second option, or both the first and second options, wherein the second option includes one or more assistance options including a request for help with one or more types of tasks, a request for one or more types of specialized personnel, and a request for one or more types of medical equipment.

3. The medical device of claim 2, wherein the one or more assistance options are customized based on the alarm triggered on the medical device.

4. The medical device of claim 2, wherein the one or more assistance options are customized based on a location of the medical device.

5. The medical device of claim 1, wherein the instructions, when executed by the at least one processing device, further cause the at least one processing device to:
route the request for assistance to one or more caregivers using a nurse call system.

6. The medical device of claim 5, wherein the request for assistance includes a location of the medical device.

7. The medical device of claim 6, wherein the location of the medical device is based on at least one of an association of the medical device with the patient, a location identification device attached to the medical device, or an input received on the display screen.

8. The medical device claim 1, wherein the instructions, when executed by the at least one processing device, further cause the at least one processing device to:
calculate a composite score using the physiological variable measurements captured by the one or more sensors; and
trigger the alarm when the composite score exceeds a threshold.

9. The medical device of claim 1, wherein the instructions, when executed by the at least one processing device, further cause the at least one processing device to:
display a shortcut on the display screen when the alarm is not triggered, wherein selection of the shortcut causes display of one or more options to submit the request for assistance, wherein the one or more options include a request for help with one or more types of tasks, a request for one or more types of specialized personnel, and a request for one or more types of medical equipment.

10. The medical device of claim 1, wherein the instructions, when executed by the at least one processing device, further cause the at least one processing device to:
display the alarm on the display screen with a first size when in an unattended mode; and
upon receipt of an acknowledgement of the alarm, switch from the unattended mode to an attended mode and display the alarm on the display screen with a second size that is smaller than the first size.

11. A method of monitoring a patient, the method comprising:
receiving measurements of a physiological variable;
triggering an alarm based on the measurements of the physiological variable; and
displaying a first option on a display screen to suppress the alarm and a second option on the display screen to submit a request for assistance.

12. The method of claim 11, further comprising:
allowing on the display screen a selection of the first option, or the second option, or both the first and second options, wherein the second option includes one or more assistance options including a request for help with one or more types of tasks, a request for one or more types of specialized personnel, and a request for one or more types of medical equipment.

13. The method of claim 12, wherein the one or more assistance options are customized based on the alarm triggered on the medical device.

14. The method of claim 12, wherein the one or more assistance options are customized based on a location of the medical device.

15. The method of claim 11, further comprising:
routing the request for assistance to one or more caregivers using a nurse call system.

16. The method of claim 15, wherein the request for assistance includes a location of the medical device.

17. The method of claim 16, wherein the location of the medical device is based on at least one of an association of the medical device with the patient, a location identification device attached to the medical device, or an input received on the display screen.

18. The method of claim 11, further comprising:
calculating a composite score using the measurements of the physiological variable; and
trigger the alarm when the composite score exceeds a threshold.

19. The method of claim 11, further comprising:
displaying a shortcut on the display screen when the alarm is not triggered, wherein selection of the shortcut causes display of one or more options to submit the request for assistance, wherein the one or more options include a request for help with one or more types of tasks, a request for one or more types of specialized personnel, and a request for one or more types of medical equipment.

20. The method of claim 11, further comprising:
displaying the alarm on the display screen with a first size when in an unattended mode; and
upon receipt of an acknowledgement of the alarm, switching from the unattended mode to an attended mode and displaying the alarm on the display screen with a second size that is smaller than the first size.
